(19) United States
(12) Patent Application Publication
(10) Pub. No.: US 2007/0207154 A1
(43) Pub. Date: Sep. 6, 2007

(54) METHOD OF MODULATING VASCULARIZATION

(76) Inventors: Martin Friedlander, Del Mar, CA (US); Wolfram Ruf, San Diego, CA (US); Michael Dorrell, San Diego, CA (US); Mattias Belting, Lund (SE)

Correspondence Address: OLSON & HIERL, LTD., 20 NORTH WACKER DRIVE, 36TH FLOOR, CHICAGO, IL, 60606, US

(21) Appl. No.: 11/578,338

(22) PCT Filed: Apr. 15, 2005
(86) PCT No.: PCT/US05/12658

§ 371 Date: Oct. 12, 2006

Related U.S. Application Data
(60) Provisional application No. 60/562,821, filed on Apr. 16, 2004.

(51) Int. Cl. A61K 39/395; A61K 38/36

(52) U.S. Cl. 424/146.1; 514/12

(57) ABSTRACT

A method of modulating vascularization in a tissue of a mammal comprises controlling a PAR signaling pathway (e.g., the PAR-1 or PAR-2 signaling pathway) in a mammalian tissue, for example, by controlling phosphorylation of tissue factor cytoplasmic domain (i.e., phosphorylation of Ser²⁵⁸ of the cytoplasmic tail of TF). In a preferred method pathological is treated by administering to a mammal suffering from pathological neovascularization, a therapeutically effective amount of a PAR signaling pathway inhibitor. Preferably the mammal is a human.

### CROSS-REFERENCE TO RELATED APPLICATIONS

This application claims the benefit of U.S. Provisional Application for Patent Ser. No. 60/562,821 filed on Apr. 16, 2004, which is incorporated herein by reference.

### GOVERNMENTAL RIGHTS

This invention was made with government support under Grant Nos. EY11254 and HL16411 from the National Institutes of Health. The government has certain rights in the invention.

### FIELD OF THE INVENTION

This invention relates to methods for modulating vascularization. In particular this invention relates to methods of modulating vascularization to stimulate or to inhibit neovascularization in a mammal by modulating the PAR-2 signaling pathway.

### BACKGROUND OF THE INVENTION

Angiogenesis, the formation of new vessels from pre-existing vasculature, plays a critical role during normal development, tissue regeneration in wound healing and post-ischemic tissue repair. Neovascularization during tumor expansion and in ischemic retinopathies are examples of angiogenesis driven disease progression (pathological neovascularization). An increased understanding of basic mechanisms that regulate physiological and pathological angiogenesis will aid the development of efficient pro- and anti-angiogenic therapies. Tissue factor (TF) is the initiator of the coagulation protease cascade that generates proteolytic fragments with potent regulatory effects on angiogenesis. TF acts as an extracellular co-receptor that activates and presents coagulation proteases for signaling through G-protein coupled, protease-activated receptors (PARs). PARs are activated through a unique mechanism that involves extracellular proteolysis of the receptor. In vitro, the TF-VIIa complex as well as factor Xa activate PAR-2. Factor Xa can also cleave PAR-1, the first identified thrombin-receptor. Factor Xa signals most efficiently in the ternary TF-VIIa-Xa complex.

In vitro data suggest that TF acts as a co-receptor in PAR signaling, but the role of the TF cytoplasmic domain in PAR signaling in vivo remains poorly defined. TF expressed by tumor cells contributes to tumor progression. TF cytoplasmic domain dependent upregulation of vascular endothelial cell growth factor (VEGF) has been suggested, although not widely confirmed, to contribute to pathological angiogenesis. In addition, TF was localized to the endothelium in malignant breast cancer, and direct inhibitors of TF has been found to suppress tumor growth and angiogenesis. PAR-1 and PAR-2 have also been implicated in angiogenesis, but in vivo data linking PAR activation by TF-initiated coagulation to angiogenesis remain sparse.

Tumor development is well known to be associated with neovascularization. For example, angiogenesis inhibitors, such as inhibitors of VEGF signaling, have been demonstrated to slow or reverse tumor growth. There is a continuing effort to discover new physiological pathways involved in tumor neovascularization and to discover new targets for inhibiting known neovascularization pathways.

Age Related Macular Degeneration (ARMD) and Diabetic Retinopathy (DR) are the leading causes of visual loss in industrialized nations and do so as a result of abnormal retinal neovascularization. Since the retina consists of well-defined layers of neuronal, glial, and vascular elements, relatively small disturbances such as those seen in vascular proliferation or edema can lead to significant loss of visual function. Inherited retinal degenerations, such as Retinitis Pigmentosa (RP), are also associated with vascular abnormalities, such as arteriolar narrowing and vascular atrophy. Retinopathy of Prematurity (ROP) is retinopathic disease associated with premature infants. ROP is the growth of abnormal blood vessels in the retina, which begins during the first few days of life and can progress rapidly (e.g., over a period of a few weeks) to cause blindness. When a baby is born prematurely, normal vessel growth may stop and new abnormal vessels may begin to grow, which over time can produce a fibrous scar tissue in the retina and can lead to retinal detachment, causing blindness. While significant progress has been made in identifying factors that promote and inhibit angiogenesis, no treatment is currently available to specifically treat ocular neovascular disease.

There is an ongoing need for methods of treating diseases involving pathological neovascularization, such as tumor development and ischemic retinopathies. The present invention fulfills this need.

### SUMMARY OF THE INVENTION

The present invention provides a method of modulating vascularization in a tissue of a mammal. The method comprises controlling a PAR signaling pathway, such as the PAR-1 or PAR-2 signaling pathway, in the tissue. The PAR signaling pathway can be controlled by controlling phosphorylation of tissue factor cytoplasmic domain in the tissue. PAR signaling pathways can be controlled by administration of a PAR signaling pathway inhibitor to the tissue.

The methods of the invention are useful for treating disease states involving pathological neovascularization, particularly in humans.

### BRIEF DESCRIPTION OF THE DRAWINGS

FIG. 1 depicts enhanced tumor growth and angiogenesis in TFΔCT mice. a, Syngeneic T241 fibrosarcoma tumor volumes and final weights at day 14 were determined in wild-type (WT) and TFΔCT mice (n=5, *t-test, p<0.05). Enhanced tumor expansion was confirmed with another tumor model, Lewis Lung Carcinoma (data not shown). Right panel: Tumor vessel density of T241 tumors based on CD31 staining. b, Ex vivo angiogenesis. Upper panel: representative light microscopy views of day 3 wild-type and TFΔCT aortic pieces embedded in Matrigel (20× original magnification); Lower panel: confocal fluorescence microscopy after staining as indicated (10× original magnification). c, Quantitation of sprouting from wild-type and TFΔCT aortas at day 3 (mean±standard error, n=74; *t-test, p<0.05). d, TF expression: Semi-quantitative PCR for TF and β-actin at day 4 in wild-type and TFΔCT aortas.

FIG. 2 depicts TF-VIIa and PDGF-BB synergy in angiogenesis. a, wild-type (WT) and TFΔCT aortic sprouting at day 3 under the indicated conditions (mean±standard error, n=10-15, *t-test, p<0.05). b, wild-type and TFΔCT aortas were incubated in serum with the addition of protease inhibitors as indicated for 3 days (*significantly different from TFΔCT control; t-test, p<0.05). c, Sprouting at day 4 from wild-type and TFΔCT aortas in EGM supplemented as indicated (mean±standard error, n=5-19, *significantly different from wild-type, t-test p<0.05). d, Enhanced sprouting from TFΔCT aortas requires PAR-2 expression: wild-type, TFΔCT, PAR-2-deficient, and TFΔCT/PAR-2-deficient aortic sprouting at day 4 (mean±standard error, n=21-37, *significantly different from wild-type, t-test, p<0.05). e, Representative aortic pieces from the respective genotype (20× original magnification).

FIG. 3 shows that the cytoplasmic-domain of tissue factor suppresses PAR-2-dependent angiogenesis. a, Upper panel: Fluorescence microscopy of day 3 TFΔCT aortic pieces co-transduced with green fluorescent protein (GFP) and human TF(1-263) or human TF(1-243) at a high virus dose (magnification 20×). Lower panel: Confocal fluorescence microscopy of TFΔCT aortic sprouts transduced with human TF(1-263) (left) or human TF(1-243) and GFP (right), and then stained as indicated (magnification 10×). b, Human TF was immunoprecipitated from detergent extracts of transduced (high dose) aortic pieces at day 4 and detected by Western blotting with polyclonal antibody to TF. c, Sprouting from wild-type (WT) and TFΔCT aortas transduced with human TF(1-263) or human TF(1-243) at a high (left panel) or low (right panel) virus dose (mean±standard error, n>13; *significantly different from control, t-test, p<0.05). d, Suppression of sprouting by human TF(1-263) requires PAR-2 expression and TF extracellular activity: wild-type, TFΔCT, and PAR-2-deficient aortas were transduced with high dose human TF(1-263), and then incubated in serum with (+anti-TF) or without antibodies to the human TF extracellular domain. Number of sprouts was determined at day 4 (mean±standard error, n>17; *significantly different from control, t-test, p<0.05).

FIG. 4 demonstrates accelerated developmental angiogenesis in TFΔCT mice. a, Representative retinas from P0 wild-type (WT), TFΔCT, PAR-2-deficient, and TFΔCT/PAR-2-deficient mice. b, A P2 wild-type retina is shown for comparison. Images were generated as montages of 4 individual images taken at 20× magnification. c, Quantitation of average vascular plexus diameter of P0 retinas from the indicated genotypes. Error bars indicate standard error of measurement.

FIG. 5 shows normal astrocyte morphology and pericyte recruitment in TFΔCT mice a, GFAP staining shows similar astrocytic templates in P0 wild-type (WT) and TFΔCT retinas (montages of images taken at 20× magnification). The left panels show a close-up (40× magnification) of the developing vascular plexus (red). b, Ki-67 staining of vascular-related nuclei showed no difference in vascular cell proliferative activity between P0 TFΔCT and P2 wild-type retinas (20× magnification). c, Quantitation of Ki-67⁺ nuclei (error bars indicate standard deviation of the mean). d, Pericyte recruitment (SMA) was similar in P0 TFΔCT, and P2 wild-type, PAR-2-deficient, or TFΔCT/PAR-2-deficient retinas (20× magnification, insets taken at 60× magnification). e. The remodeled superficial vascular plexus architecture was also similar in P6 TFΔCT and P8 wild-type retinas (montages of images taken at 10× magnification). In all cases, vessels were visualized by staining with isolectin griffonia simplicifolia.

FIG. 6 illustrates TF phosphorylation and PAR-2 expression in ocular neovascularization. a, Iris specimen #1 stained with TF cytoplasmic domain Ser²⁵⁸ phosphorylation specific antibody (P-TF) or polyclonal antibody to PAR-2 (confirmed to block PAR-2 cleavage). To show specificity, stainings were also performed in the presence of the peptide immunogen (magnification 20×). b, c, Additional independent iris specimens from diabetic patients demonstrate TF phosphorylation in pathological vessels (b, montage of images with 10× magnification, inset 40× magnification. c, 20× magnification). d, Iris specimen from a non-diabetic glaucoma patient shows absence of phosphorylated TF. e-h, Specimen from a patient with clinically diagnosed proliferative diabetic retinopathy (10× magnification). e, Staining with polyclonal anti-TF extracellular domain antibody (TF) shows widespread expression of TF in the retina. Pathological (arrow), but not normal (arrowheads), vessels show staining for phosphorylated TF (magnification 40×). f, PAR-2 staining is observed within and adjacent to abnormal retinal neovessels (magnification 40×). g,h, Phosphorylated TF is localized in abnormal retinal neovessels, confirmed by co-staining with LM609 antibody specific for integrin αᵥβ₃ (g, magnification 40×, h, magnification 20×).

FIG. 7 graphically illustrates the extent of vascular obliteration (top panel) and neovascular tuft formation (bottom panel) in neonatal mice exposed to hyperoxia (wt=wild type mice; Par-1=PAR-1 deficient mice; dCTPar1=PAR-1 deficient mice also having the TFΔCT mutation).

FIG. 8 graphically illustrates the extent of vascular obliteration (top panel) and neovascular tuft formation (bottom panel) in neonatal mice exposed to hyperoxia (wt=wild type mice; dCT=mice having the TFΔCT mutation; PAR-2 PAR-2 deficient mice; dCT/PPAR-2=PAR-2 deficient mice also having the TFΔCT mutation; P15=postnatal Day 15; P17=postnatal Day 17).

FIG. 9 graphically illustrates the extent of vascular obliteration (top panel) and neovascular tuft formation (bottom panel) in neonatal wild-type mice exposed to hyperoxia and treated with an inhibitor of PAR signaling versus a control substance (FVIIai=active site mutated factor VII treated mice; PBS=control mice treated only with phosphated saline buffer).

### DETAILED DESCRIPTION OF PREFERRED EMBODIMENTS

As used herein and in the appended claims, the term therapeutically effective amount, in reference to an inhibitor of a PAR signaling pathway, such as the PAR-2 signaling pathway or the PAR-1 signaling pathway, including inhibitors of TF-VIIa signaling, PDGF receptor β signaling, and tissue factor cytoplasmic domain phosphorylation inhibitors, means an amount of inhibitor, which when administered to a mammal suffering from pathological neovascularization, reduces of eliminates undesirable neovascularization. Administration can be in a single dose or in multiple doses over a set period of time or indefinitely. A therapeutically effective amount can readily be readily determined by one of ordinary skill in the medical arts.

PAR signaling can impact neovascularization in mammalian tissues. Phosphorylation of tissue factor cytoplasmic domain (i.e., phosphorylation of Ser²⁵⁸ of the cytoplasmic tail of TF) stimulates PAR expression in tissues where such phosphorylation occurs, leading to pathological neovascularization. Accordingly, control of PAR signaling, for example by phosphorylation of the tissue factor cytoplasmic domain can be utilized to modulate vascularization (i.e., to promote or inhibit angiogenesis).

Modulation of neovascularization by the PAR signaling pathways involves a number of factors and intersects with other signaling pathways, including the TF-VIIa complex signaling, factor Xa signaling, and platelet-derived growth factor (PDGF) receptor β signaling. A method for modulating vascularization in a mammalian tissue comprises controlling PAR signaling in the tissue, preferably controlling the PAR-2 signaling pathway.

Pathological neovascularization in a mammal is treated by administering to a mammal suffering from pathological neovascularization, a therapeutically effective amount of a PAR signaling pathway inhibitor. Preferably the mammal is a human. Non-limiting examples of preferred inhibitors of PAR signaling pathways include inhibitors of TF-VIIa signaling, inhibitors of PDGF receptor β signaling, and inhibitors of tissue factor cytoplasmic domain phosphorylation.

One preferred method aspect of the present invention comprises administering to a mammal suffering from pathological neovascularization a therapeutically effective amount of a TF-VIIa signaling inhibitor. Non-limiting examples of preferred TF-VIIa signaling inhibitors include active site inhibited VIIa (VIIai), nematode anticoagulant peptide c2 (NAPc2), antibodies specific for factor VIIa and antibodies specific for TF-VIIa complex, and the like.

Another preferred method aspect of the present invention comprises administering to a mammal suffering from pathological neovascularization a therapeutically effective amount of a PDGF receptor β signaling inhibitor. Non-limiting examples of PDGF receptor β signaling inhibitors include antibodies specific for PDGF-BB, and the like.

Yet another preferred method aspect of the present invention comprises administering to a mammal suffering from pathological neovascularization a therapeutically effective amount of a tissue factor cytoplasmic domain phosphorylation inhibitor.

Non-limiting examples of disease states involving pathological neovascularization, which can be treated by the methods of the present invention include cancers involving tumor development (e.g., in breast cancer, lung cancer, and the like) and ischemic retinopathic diseases, such as diabetic retinopathy, age related macular degeneration, retinopathy of prematurity, and the like.

### EXAMPLE 1

Mouse strains and reagents. The TFΔCT mouse strain, lacking the 18 carboxyl-terminal residues of the TF cytoplasmic domain, and PAR-2-deficient mice (kindly provided by P. Andrade-Gordon, Johnson & Johnson Pharmaceutical Research & Development) were back-crossed to yield >90% homogeneity with the C57/BL6 genetic background. TFΔCT/PAR-2-deficient double knock-outs were generated by interbreeding after five generations of backcrossing. The source of reagents was as follows: Matrigel (Beckton & Dickinson), endothelial cell growth medium (EGM, Clonetics), DMEM (GIBCO), growth factors (R&D Systems), TOPRO and isolectin griffonia simplicifolia (Molecular Probes), antibodies to CD31 (Santa Cruz) and to SMA and GFAP (SIGMA), Ki-67 (NOVO Laboratories). Goat antibody and monoclonal antibodies to TF, VIIai, hirudin, VIIa were previously described by Riewald, M., and Ruf, W. Proc. Natl. Acad. Sci. USA 98, 7742-7747 (2001). NAPc2 and NAP5 were kindly provided by G. Vlasuk (Corvas International). Adenoviral constructs of human TF(1-263) and human TF(1-243) were described by Dorfleutner, A., and Ruf, W. Blood 102, 3998-4005 (2003). The proteinase-activated receptor 2 is induced by inflammatory mediators in human endothelial cells and Ad5 serotype vectors coexpressing GFP were similarly generated.

Tumor growth. All animal tests were approved by the Institutional Animal Care and Use Committee of The Scripps Research Institute. 4×10⁵ T241 fibrosarcoma cells were injected s.c. into wild-type and TFΔCT mice aged 7-9 weeks and >97% C57BL/6. Tumor volumes and final weights at day 14 were determined followed by embedding of the tumors in OCT. Ten μM cryosections were fixed with acetone, stained for CD31, and vessel density/microscopic field was determined by fluorescence microscopy from 6-8 sections of two tumors each from wild-type and TFΔCT mice.

Angiogenesis assays. The ex vivo angiogenesis assay was adopted from the rat aortic sprouting model described by Masson et al. Biol. Proced. 4, 24-31 (2002) and Nicosia et al. Lab Invest. 63, 115-122 (1990). Thoracic aortas from 8-11 week old wild-type, TFΔCT, PAR-2-deficient, and TFΔCT/PAR-2-deficient mice of either sex were embedded in Matrigel, and overlaid with EGM supplemented with 5% serum, growth factors, or inhibitors at the following concentrations: VEGF, bFGF, and PDGF: 20 ng/ml; hirudin: 500 nM, VIIai: 100 mM, NAPc2: 200 μM, NAP5: 1 μM, VIIa: 50 nM. In most cases, the number of aortic sprouts was determined at day 3 and 4 without knowledge of the genotype. Aortic ring RNA was isolated by Trizol (Invitrogen) extraction using standard procedures, digested with DNAaseI, followed by RT-PCR for β-actin and TF. Aortic pieces were transduced with adenovirus constructs for full-length human TF(1-263) or truncated human TF(1-243) in serum-free DMEM for about 20 to 24 hours prior to embedding into the sprouting assay. Tests used 2 different virus doses, referred to as high (1.1×10¹⁰ virus particles/ml) and low (5×10⁹ particles/ml). For confocal fluorescence microscopy, aortic pieces with a minimum of surrounding Matrigel were fixed with 4% paraformaldehyde and methanol, incubated with primary and secondary antibodies (24 hours each), and mounted in anti-fade medium (Vector laboratories). Alternatively, cryosections of OCT-embedded aortas were fixed with acetone and stained as described above.

To evaluate neonatal angiogenesis, retina whole mounts were prepared, and angiogenesis was quantified as described by Dorrell et al. Invest. Opthalmol. Vis. Sci. 43, 3500-3510 (2002) The number of retinas and different litters used for the respective genotype were: wild-type (20 retinas from 6 litters), TFΔCT (24 retinas from 5 litters), PAR-2-deficient (10 retinas from 3 litters), and TFΔCT/PAR-2-deficient (16 retinas from 4 litters). Dissected retinas were fixed in 4% paraformaldehyde followed by methanol, incubated in primary antibody or fluorescence conjugated isolectin griffonia simplicifolia overnight, followed by secondary antibody incubation and mounting. Retinas were imaged using the same magnification, resolution, and intensity parameters. Images were assembled as single retina montages and the diameter of vascularization was quantified using LaserPix software (BioRad) (6 diameter measurements from 1, 2, 3, 4, 5, and 6 o'clock+two random diameter measurements). Total numbers of vascular-related Ki-67⁺ nuclei were determined by focusing within the vascular plane. Focusing within this plane eliminated proliferating neuronal cells from the count.

Analysis of ocular specimens. All tests using human tissues were performed according to approved human protocols and with permission from informed patients. Specimens of iris, already scheduled to be removed for clinical reasons, were immediately immersed in 20% sucrose at about 4° C. prior to creation of frozen sections. The specimen of neovascular retina was obtained from the San Diego eye bank. This eye was obtained from a patient clinically diagnosed with diabetic retinopathy for over 25 years. After dissection, the retina was fixed with 4% PFA overnight at about 4° C., followed by cryoprotection in 20% sucrose, and cryosectioning. Frozen sections were processed for primary antibodies and detected with secondary antibodies conjugated either to alexa 488, alexa 568, or alexa 633 (Molecular Probes) for confocal microscopy. Mouse antibodies to CD31 (Biocare Medical, 1:50) and to integrin αᵥβ₃ (LM609, 1:500), rhodamine conjugated Ulex Europaeus Agglutinin 1 (Vector Laboratories, 1:1000), rabbit antibodies to the TF extracellular domain (R4563, 25 μg/ml), to Ser²⁵⁸ phosphorylated TF cytoplasmic domain (R6936, 25 μg/ml), and to PAR2 (R6797, 25 μg/ml) were used. The peptides used as immunogens for R6936 and R6797 were added at about 50 μg/ml to demonstrate specificity.

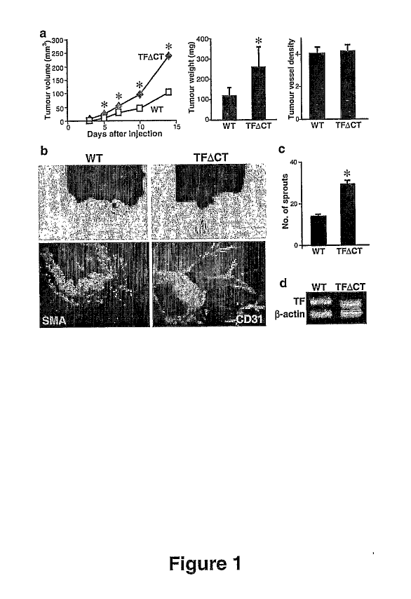

TF cytoplasmic domain deletion enhances angiogenesis. To evaluate the role of the TF cytoplasmic domain in tumor angiogenesis, we studied the growth of syngeneic tumors in TF cytoplasmic domain-deleted mice (TFΔCT) in comparison to wild-type littermate offspring (FIG. 1, Panel a). Tumor expansion and final tumor weight were approximately two-fold enhanced in TFΔCT as compared with wild-type mice. However, tumors from wild-type and TFΔCT mice displayed similar end-stage vessel density (FIG. 1, Panel a), consistent with the notion that tumor expansion followed increased blood supply and that the tumor cells established a similar neovasculature in these mice. However, these data did not exclude the possibility that TF expressed by host stromal cells contributed to the accelerated angiogenesis in TFΔCT mice. To directly analyze the regulatory role of the TF cytoplasmic domain in vascular cells under defined conditions, we employed the aortic ring assay which was carried out in the presence of autologous mouse serum to stimulate angiogenesis. Microvessel sprouting from TFΔCT mouse aortas was enhanced 2-fold relative to wild-type aortas (FIG. 1, Panel b, c). Aortic sprout cells were primarily endothelial, as shown by positive staining for CD31 and negative staining for smooth muscle cell actin (SMA) (FIG. 1, Panel b). We observed similar TF expression levels in sprouting TFΔCT and wild-type aortas (FIG. 1, Panel d), indicating that loss of the TF cytoplasmic tail rather than de-regulated TF expression causes accelerated endothelial cell sprouting in TFΔCT mice.

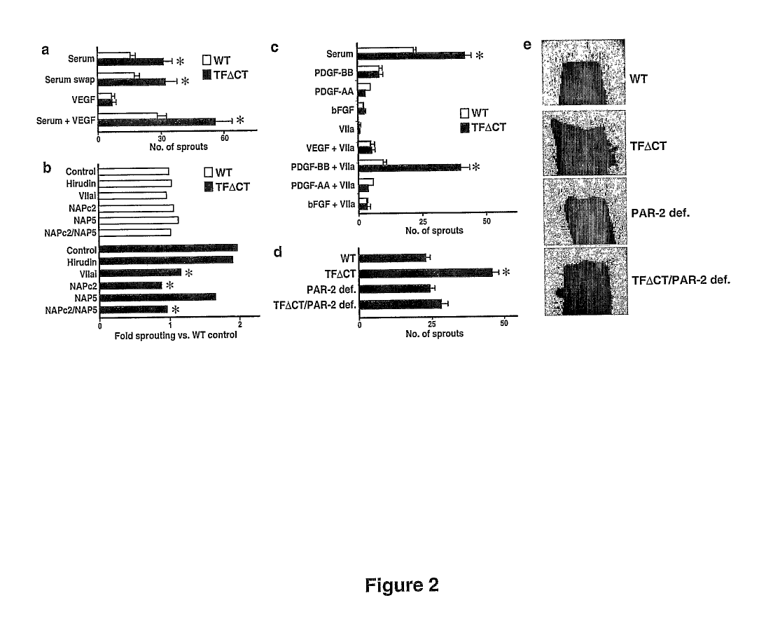

Because TF cytoplasmic domain signaling was implicated in regulating VEGF expression by tumor cells, we tested whether wild-type aortas exhibit reduced sprouting due to a relative deficiency in VEGF. Supplementing serum with VEGF did not abolish the difference in sprouting from TFΔCT versus wild-type aortas. However, aortas stimulated with VEGF in the absence of serum showed very limited sprouting, demonstrating that serum was required for accelerated angiogenesis from TFΔCT aortas (FIG. 2, Panel a). Sprouting from wild-type aortas in the presence of TFΔCT mouse serum (serum swap) was not enhanced, indicating that TF expressed by TFΔCT vascular cells, rather than a serum factor or increased levels of circulating TF, confers the pro-angiogenic phenotype (FIG. 2, Panel a).

TF-VIIa signaling accelerates angiogenesis in TFΔCT aortas. The serum dependence of the TFΔCT sprouting phenotype suggested that genetic ablation of the TF cytoplasmic tail may unmask coagulation factor pro-angiogenic activity. The inhibitory effects of blocking coagulation proteases in the aortic ring sprouting model was investigated (FIG. 2, Panel b). Inhibition of thrombin by hirudin, as well as inactivation of Xa by the nematode anticoagulant peptide (NAP) 5 had no effect on sprouting, which excluded contributions from proteases downstream in the coagulation cascade. Active site inhibited VIIa (VIIai), a high affinity competitive antagonist that blocks TF-VIIa complex formation, as well as the nematode inhibitor NAPc2, which inhibits TF-VIIa by forming a trapped TF-VIIa-Xa complex, reversed the TFΔCT sprouting phenotype, but did not influence sprouting from wild-type aortas. These results demonstrate that the TF cytoplasmic domain negatively regulates TF-VIIa protease signaling.

To directly analyze the role of factor VIIa (“VIIa”) in angiogenesis, serum was replaced with VIIa in the aortic ring model. TF-VIIa inefficiently induced sprouting from both wild-type and TFΔCT aortas (FIG. 2, Panel c). Because endothelial cell sprouting is typically dependent on growth factor signaling, we further characterized sprouting from TFΔCT aortas in the presence of defined pro-angiogenic growth factors, i.e. VEGF, platelet-derived growth factor (PDGF) AA, PDGF-BB, or basic fibroblast growth factor (bFGF). None of these factors promoted substantial sprouting, which is consistent with previous data, and the pro-angiogenic phenotype of TFΔCT was not apparent in the presence of any of the growth factors alone. However, combining TF-VIIa with PDGF-BB selectively recapitulated the pro-angiogenic phenotype of TFΔCT observed under serum conditions (FIG. 2, Panel c). No evidence of an additive effect of PDGF-BB and VIIa on sprouting from wild-type aortas was observed, as described for fibroblast migration. PDGF-AA is a selective agonist for PDGF receptor a, but cannot activate PDGF receptor β. Because VIIa did enhance angiogenesis in the presence of PDGF-BB, but not PDGF-AA, TF-VIIa signaling appears to synergize with PDGF receptor β signaling when negative regulatory control by the TF cytoplasmic domain is lost.

TF-VIIa signaling crosstalk with PAR-2 regulates angiogenesis. TF-VIIa-dependent PAR-2 activation accelerates angiogenesis in TF cytoplasmic domain deleted mice in synergy with PDGF-BB. Aortic ring sprouting in TFΔCT/PAR-2-deficient double transgenic mice was reverted to wild-type levels (FIG. 2, Panel d), demonstrating that loss of the TF cytoplasmic domain leads to PAR-2-dependent accelerated angiogenesis. The lack of a phenotype in PAR-2-deficient aortas further indicates that the TF cytoplasmic tail is highly efficient in suppressing PAR-2's proangiogenic effects, which is also supported by the finding that TF-directed inhibitors (VIIai and NAPc2) did not reduce sprouting from wild-type aortas (FIG. 2, Panel b).

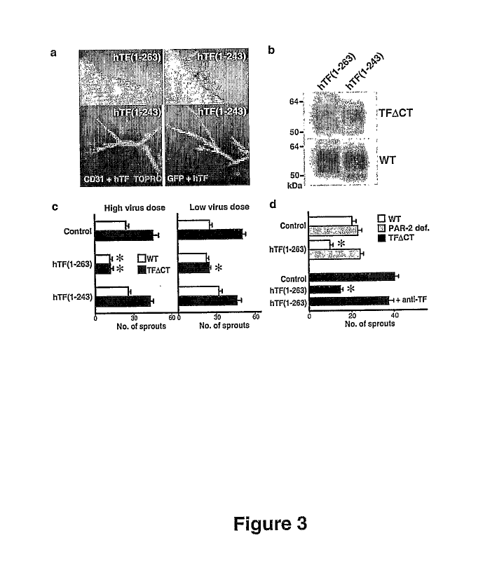

In order to exclude that the phenotype of TFΔCT mice is unrelated to TF cytoplasmic domain signaling, either full-length, human TF(1-263) or, as a control, cytoplasmic domain-deleted, human TF(1-243) was restored by adenoviral transduction in wild-type or TFΔCT aortas. Co-expression of green fluorescent protein (GFP) and staining with human specific anti-TF antibodies showed that the migration of endothelial sprout cells into the surrounding matrigel Was suppressed by human TF(1-263), but not human TF(1-243) (FIG. 3, Panel a). Co-localization of human TF with CD31 further identified endothelial cells as targets for adenoviral transduction. Expression levels were determined in extracts from aortic ring assays by detecting human TF in Western-blots, confirming equal expression levels of both forms of TF (FIG. 3, Panel b). At the higher virus dose, human TF(1-263) suppressed sprouting from both wild-type and TFΔCT aortas (FIG. 3, Panel c, left), whereas with less administered virus, TFΔCT sprouting was selectively reversed to wild-type levels (FIG. 3, Panel c, right). In all cases, truncated human TF(1-243) had no effect, showing that suppression is dependent on the TF cytoplasmic tail (FIG. 3, Panel c). These data support the concept that, when introduced at the appropriate levels, the TF cytoplasmic domain can restore negative regulatory control of PAR-2 signaling in angiogenesis.

In order to gain further insight into the mechanism of how the TF cytoplasmic domain suppresses PAR-2 signaling in angiogenesis, the reversal of the proangiogenic phenotype of TFΔCT aortas was examined to determine whether it required signaling and extracellular protease assembly with the introduced human TF. Blockade of the extracellular domain of TF(1-263) with human specific monoclonal antibodies prevented the reversal of the enhanced sprouting phenotype of TFΔCT mice (FIG. 3, Panel d). The involvement of PAR-2 was addressed by capitalizing on the finding that expression of high levels of human TF(1-263) suppressed sprouting from wild-type aortas. Equivalent virus doses did not reduce sprouting from PAR-2-deficient aortas, demonstrating that the suppressive function of the TF cytoplasmic domain requires PAR-2 expression (FIG. 3d). Collectively, these data demonstrate that negative regulatory control of angiogenesis by the TF cytoplasmic domain specifically occurs in the context of PAR-2 signaling.

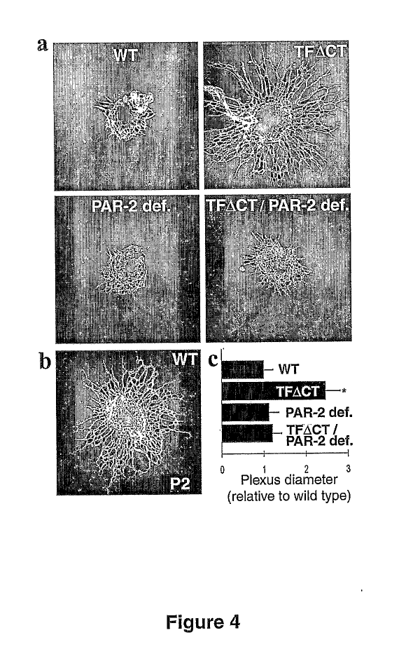

The TF cytoplasmic domain regulates physiological angiogenesis. To further address the role of the TF cytoplasmic domain in vivo, physiological angiogenesis in the neonatal retina, which develops a vascular network emanating from the optic disc in a stereotypical manner, was investigated. In neonatal mice, the superficial vascular plexus diameter of TFΔCT was twice that of wild-type mice, demonstrating that the TF cytoplasmic tail negatively regulates in vivo angiogenesis during postnatal development (FIG. 4, Panel a). The extent of vascularization in newborn TFΔCT retinas was comparable to 2 day old (P2) wild-type retinas (FIG. 4, Panel b). Consistent with the data in the aortic ring assay, retinas from neonatal PAR-2-deficient as well as TFΔCT/PAR-2-deficient double transgenic mice showed age appropriate vascularization (FIG. 4, Panel a). The evaluation of at least ten retinas derived from at least three different pregnancies for each genotype confirmed the consistency of the observed phenotype of TFΔCT mice and of its reversal by simultaneous deletion of PAR-2 (FIG. 4, Panel c).

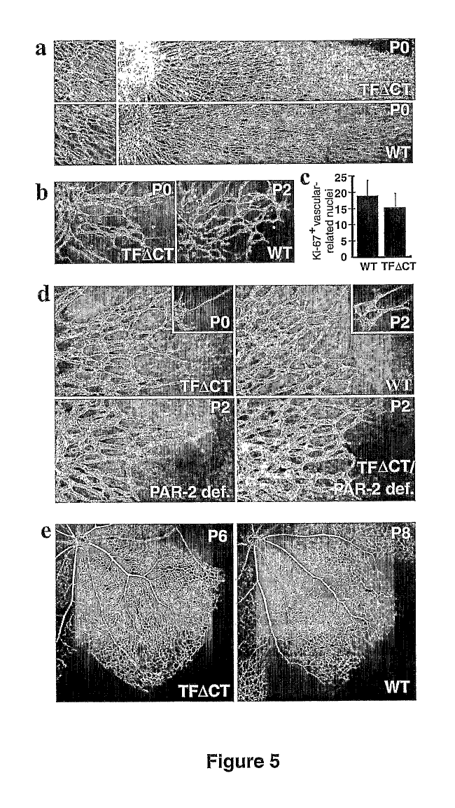

Vascular cell-specific localization of TF in TFΔCT retinas was difficult to evaluate, because of the prominent expression of TF by astrocytes, an established TF-expressing cell type in the CNS, as well as potential TF expression by the underlying nerve fibers. Glial fibrillary acidic protein (GFAP) staining for astrocytes showed that astrocytes similarly extended to the periphery of retinas from wild-type and TFΔCT newborn mice with no apparent difference in the staining pattern (FIG. 5, Panel a). Thus, vascular development did not indirectly follow accelerated developmental astrocyte migration in TFΔCT retinas. Vascular apoptosis is infrequently observed in wild-type mice at this stage of development, and protection from apoptosis is an unlikely cause for accelerated angiogenesis in TFΔCT mice. Enhanced vascular development may result from increased cell proliferation, but proliferating vascular cells based on Ki-67 staining are present in comparable number in both newborn TFΔCT and P2 wild-type retinas (FIG. 5, Panel b, c). The plexus of P0 TFΔCT retinas appeared to be more extended in comparison with P2 wild-type retinas (FIG. 4, Panel a, b). This reflects enhanced endothelial cell migration, consistent with the scaffolding function of PAR-2 to localize the MAP kinase pathway at the leading edge of migrating cells. TF is expressed in angiogenic endothelial cells associated with malignant breast tumors. In vitro studies have shown direct effects of PDGF-BB on primary endothelial cell migration and cord/tube formation via activation of PDGF receptor-β, which is detectable on capillary endothelial cells in vivo.

PDGF-BB signaling is also important to the recruitment and expansion of mural cell/pericyte populations that stabilize and regulate remodeling of the vascular architecture. Moreover, complete deletion of the TF gene caused defective vascular remodeling of the embryonic vascular plexus in the yolk sac with associated reduction in pericyte recruitment. The close association between endothelial and mural cells during angiogenic sprouting makes it challenging to distinguish between autocrine effects of PDGF-BB on endothelial cells and secondary, paracrine effects on recruited mural cells. Using SMA staining as a pericyte-specific marker, a similar staining pattern was observed in the retina vasculature from newborn TFΔCT and P2 wild-type mice (FIG. 5, Panel d). Pericyte staining in each case extended to the tips of the sprouts (FIG. 5, Panel d, insets). The vascular plexus of P2 PAR-2-deficient or TFΔCT/PAR-2-deficient mice were indistinguishable from P2 wild-type mice, excluding the possibility that defective vascular development in PAR-2-deficient mice was not apparent at earlier times. Pericytes play important roles in remodeling the developing retinal vascular plexus. The equivalently expanded superficial vascular plexus of P6 TFΔCT and P8 wild-type retinas also show comparable capillary network density, distribution of arteries or veins, and pattern of SMA staining (FIG. 5, Panel e). These similarities at later stages of retina vascularization argue against altered pericyte function. Accelerated vascular development in TFΔCT retinas persisted at least until day P6 at which time premature endothelial cell sprouting into the deeper layers of the retina was observed. Collectively, these data are consistent with a phenotype of accelerated endothelial cell migration in the development of the superficial vascular plexus, rather than abnormal pericyte recruitment in TFΔCT mice.

TF cytoplasmic domain phosphorylation in neovascular eye disease. In order to examine if TF phosphorylation occurs in other cases of pathological angiogenesis, specimens of neovascularized iris that were removed from diabetic patients were analyzed.

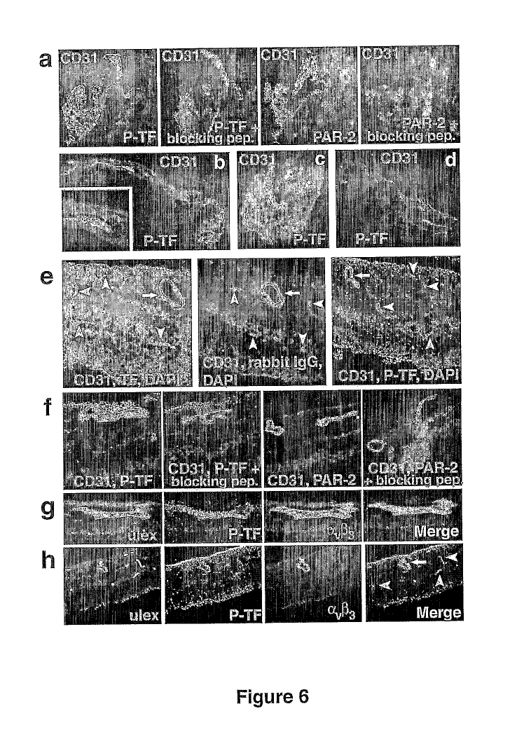

The TF cytoplasmic domain is typically non-phosphosphorylated in endothelial cells. Phosphorylation may release negative regulatory effects of the TF cytoplasmic domain and thus promote pathological angiogenesis. Indeed, staining with an antibody that specifically recognizes TF phosphorylated at Ser²⁵⁸ identified TF cytoplasmic domain phosphorylation only at sites of neovascularization in specimens from six different patients (FIG. 6, Panel a, b, c). TF phosphorylation in these pathological vessels colocalized with PAR-2 expression (FIG. 6, Panel a), supporting a role for uncontrolled PAR-2 signaling during pathological neovascularization. Importantly, phosphorylated TF and PAR-2 staining were not observed in control iris samples from a glaucoma patient with no history of diabetes or pathological neovascularization (FIG. 6, Panel d).

TF phosphorylation and PAR-2 upregulation were also observed specifically in neovessels in a retina obtained from a patient with diabetic retinopathy. Staining with an antibody to the TF extracellular domain demonstrated widespread expression of TF in glial and neuronal cell types, mature vessels (FIG. 6, Panel e, arrowheads) and at sites of neovascularization (FIG. 6, Panel e, white arrow). However, TF phosphorylation was only observed in dilated pathological vessels (FIG. 6, Panel e). Staining of phosphorylated TF in pathological vessels was completely eliminated by competition with the antigenic peptide (FIG. 6, Panel a, f). Non-specific punctuate staining that was incompletely competed by the immunogen was sometimes observed in the inner and outer limiting membranes, regions notorious for non-specific staining by different antibodies in retina specimens.

TF phosphorylation was not observed on normal, mature retinal vessels, supporting a specific role for TF phosphorylation during pathological neovascularization. In serial sections, pronounced PAR-2 expression was observed specifically in the same vessels where TF phosphorylation was observed (FIG. 6, Panel f). To ensure that TF phosphorylation was specific to neovessels, we stained diabetic retinas for integrin αᵥβ₃, a known marker of vascular proliferation (FIG. 6, Panel g, h). Phosphorylated TF consistently co-localized with αᵥβ₃-positive neovessels, while normal retinal microvasculature was negative for both (FIG. 6, Panel g, h).

### EXAMPLE 2

This example illustrates the role of p53 in tissue factor signaling. Retinas from TFΔCT and TFΔCT/p53 double mutant mice were examined at postnatal Day 0 (P0) and at postnatal Day 6 (P6). The retinal phenotype of tissue factor cytoplasmic tail deleted (TFΔCT) neonatal mouse, which exhibits accelerated vascularization of retina during development, was reverted in TFΔCT/p53 double mutant mice, indicating that p53, originally found as a tumor suppressor protein, interacts with TF signaling.

### EXAMPLE 3

This example illustrates the effects of hyperoxia on TFΔCT, TFΔCT/PAR-2 and TFΔCT/PAR-1 mice. The role of tissue factor cytoplasmic tail and protease activated receptors (PAR) 1 and 2 in the pathological angiogenesis was studied using the mouse model for oxygen induced retinopathy (OIR). Neonatal wild type (wt), TFΔCT, PAR-2-deficient and PAR-1-deficient mice (kindly provided by Johnson & Johnson Pharmaceutical Research & Development), as well as TFΔCT/PAR-2 and TFΔCT/PAR-1 deficient double mutants, were exposed to hyperoxia (75% oxygen) at P7 for 5 days. Since TFΔCT have an accelerated rate of retinal vascularization, mice were placed in hyperoxia at PS when retinal vascularization is comparable to a P7 wild type. At P12 and P17 (immediately and 5 days after return to normoxia, respectively), retinas were dissected, fixed in 4% PFA, and incubated with fluorescein conjugated isolectin Griffonia sinplicifolia. Retinas were imaged using confocal microscopy and areas of obliteration and neo-vascular tufts were quantified.

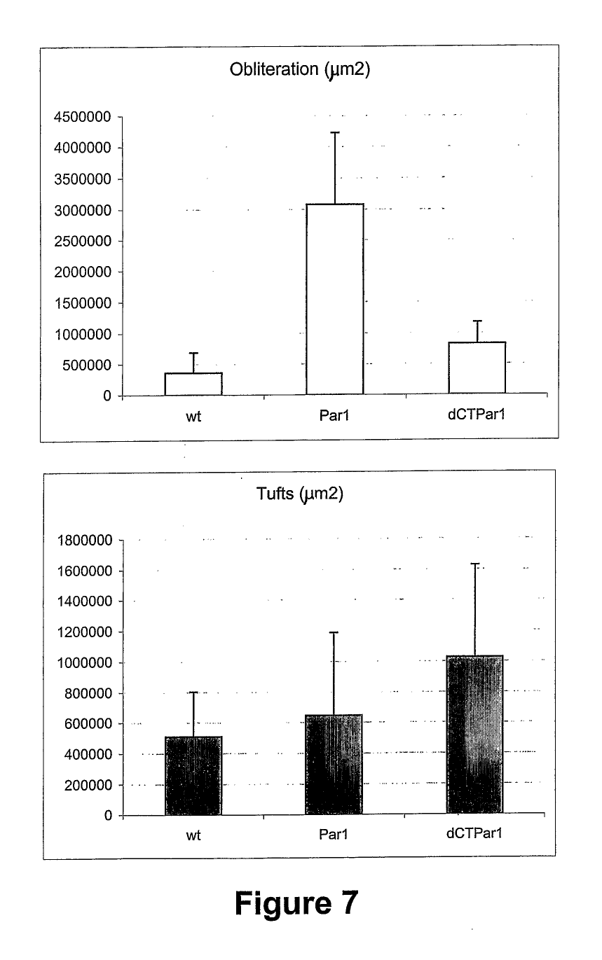

Immediately after hyperoxia exposure, the extent of obliteration was similar in all mice. FIG. 7 graphically compares TFΔCT mice and TFΔCT/PAR-1 deficient double mutants to wild-type mice. The upper panel of FIG. 7 shows the wild-type mice and double mutants had similar levels of vascular obliteration, whereas in PAR-1 deficient mice, revascularization of obliteration area was significantly delayed compared to wild-type mice. In TFΔCT/PAR-1 deficient double mutants, this delay of revascularization, as evidenced by neovascular tuft formation (FIG. 7, bottom panel), was partially reverted.

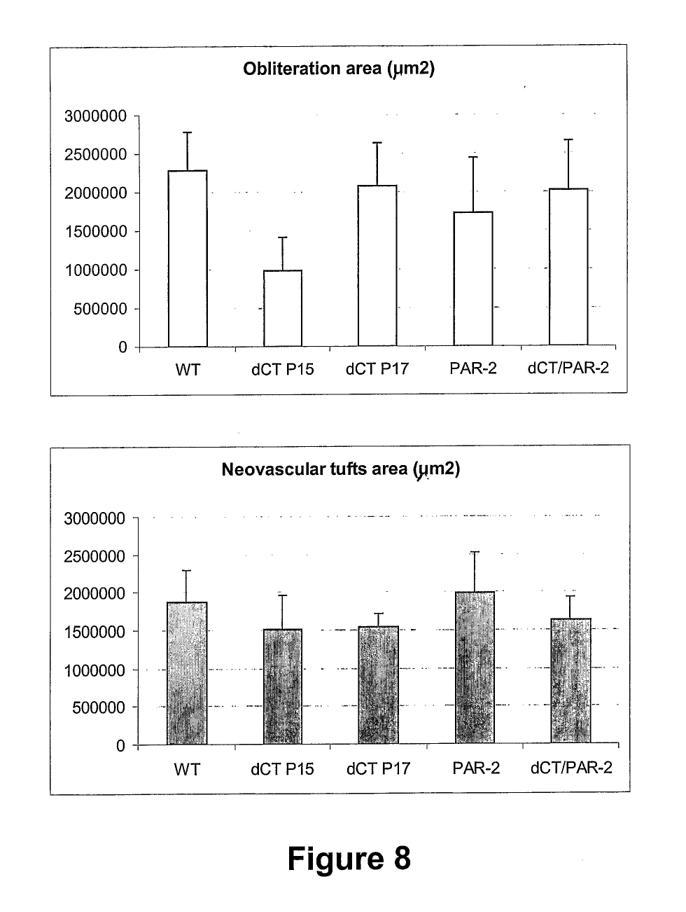

FIG. 8 graphically compares TFΔCT mice and TFΔCT/PAR-2 deficient double mutants to wild-type mice. The upper panel of FIG. 8 shows that at p17, TFΔCT mice demonstrated significantly smaller retinal vascular obliteration areas than wild-type mice, demonstrating that loss of TF cytoplasmic tail results in enhanced revascularization of obliterated areas. TFΔCT/PAR-2 deficient double mutants reverted the TFΔCT phenotype, showing that PAR-2 signaling is regulated by cytoplasmic tail of tissue factor in pathological angiogenesis. No significant alteration in the extent of obliteration was observed in PAR-2 knockouts. In contrast to differences observed in the revascularization of obliteration area, no significant differences in the formation of neovascular tufts was observed in any of transgenic mice compared to wild-type mice at P17 (FIG. 8, bottom panel).

### EXAMPLE 4

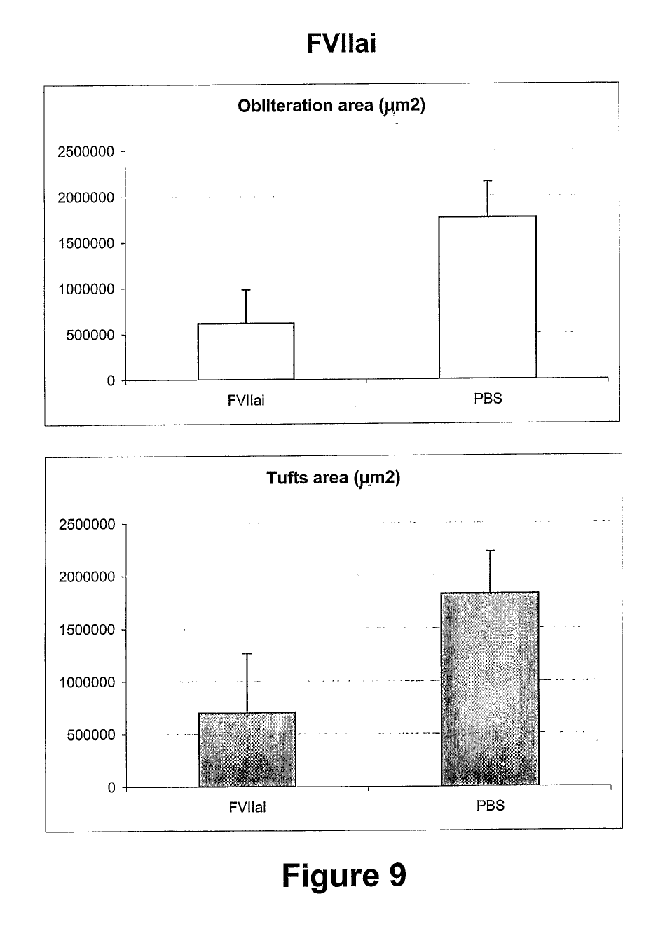

This example illustrates the effect of injections of a PAR signaling inhibitor (active-site inhibited factor VII (FVIIai) prepared by the method described by Dickinson and Ruf, J. Biological Chem., 1997; 272:19875-19879) in mice in the OIR model. To further study role of TF signaling in the mouse model of OIR, recombinant active site mutated factor VII inhibitor (FVIIai), which has higher affinity to TF than naturally occurring factor VII, was injected intravitreally immediately after mice were returned normoxia. Contralateral control eyes in FVIIai injected mice were injected with PBS as a control. Retinas were analyzed 4 days after injections. FVIIai injection enhanced revascularization of the obliteration area (FIG. 9, upper panel), whereas formation of neo-vascular tufts was reduced (FIG. 9, bottom panel).

Discussion

Angiogenesis is an important component of the pathology observed in cancer, neovascular eye diseases and arthritis where activation of coagulation is prevalent. In fact, coagulation may indirectly support angiogenesis by multiple effects, including the generation of a transitional fibrin rich extracellular matrix, the release of pro- and anti-angiogenic factors from activated platelets, and thrombin signaling through endothelial cell PAR-1. The present data provide novel and unexpected insight into how coagulation signaling regulates angiogenesis by demonstrating that PAR-2 signaling is tightly controlled by the TF cytoplasmic domain. Genetic deletion of the TF cytoplasmic domain results in accelerated physiological and pathological angiogenesis. Thus, loss of negative regulatory control by the TF cytoplasmic domain is a novel pathway by which pro-angiogenic signaling of PAR-2 can be turned on.

Whereas PAR-1 is constitutively expressed in endothelial cells, PAR-2 is specifically upregulated upon inflammatory cytokine stimulation which also induces TF. However, TF expression is synergistically enhanced by concomitant VEGF signaling in endothelial cells. Expression of TF and PAR-2 and the functionality of the TF-PAR-2 signaling pathway thus depend on the availability of both angiogenic growth factors and inflammatory cytokines. Inflammatory cytokine production by recruited monocyte/macrophages is recognized as important for angiogenesis and collateral growth of vessels. These adaptive processes share similarities with wound repair that is typically associated with activity of the innate immune system to clear pathogens from injured tissues. Accelerated angiogenesis during wound repair when there is concomitant inflammation may be the physiological function of the TF-PAR-2 signaling pathway and, thus, explain the evolutionary conservation of the TF cytoplasmic domain structure and regulatory elements in vertebrates.

Physiological response pathways frequently cause pathology when negative regulatory control mechanisms are lost. The TF cytoplasmic domain is the target for posttranslational modifications by Ser phosphorylation through PKCα-dependent pathways in endothelial cells. TF is primarily non-phosphorylated and palmitoylation suppresses agonist-induced phosphorylation. In addition, PAR-2, but not PAR-1, activation leads to TF cytoplasmic domain phosphorylation in endothelial cells. Thus, loss of palmitoylation in conjunction with upregulation of PAR-2 determines the degree of TF cytoplasmic domain phosphorylation. This concept is supported by in vivo data from diabetic eye tissue; a striking colocalization of upregulated PAR-2 with phosphorylated TF was observed only in neovessels. Thus, phosphorylation of TF is the probable mechanism that switches off negative regulatory control to promote pathological PAR-2-dependent angiogenesis.

TF-PAR-2 signaling selectively synergized with PDGF-BB and not VEGF, bFGF, or PDGF-AA in TFΔCT aortas. PDGF-BB is readily available either by release from activated platelets in the context of local coagulation or by synthesis from sprouting endothelial cells. While VEGF-targeted anti-angiogenic therapy appears efficacious in certain diseases, additional benefit may be obtained from combination therapy with molecules that target alternative and cooperative pathways. For example, inhibiting PDGF receptors has synergistic benefit in combination with VEGF-directed approaches. Since PDGF-BB signaling is crucial in stabilizing pericyte recruitment and mature vessel architecture, generalized PDGF receptor blockade has obvious limitations. Indeed, reduced vascular pericyte density as a result of endothelium-specific PDGF-BB ablation causes microvascular angiopathy in mice.

Numerous variations and modifications of the embodiments described above can be effected without departing from the spirit and scope of the novel features of the invention. For example, ischemia can be treated by a systemic or local administration of a therapeutically effective amount of TF having a phosphorylated cytoplasmic domain to a patient in need of such treatment. No limitations with respect to the specific embodiments illustrated herein are intended or should be inferred.

We claim:

1. A method of modulating vascularization in a tissue of a mammal, which comprises controlling a protease activated receptor (PAR) signaling pathway in said tissue.

2. The method of claim 1 wherein the controlling comprises inhibition of PAR-2 signaling pathway.

3. The method of claim 1 wherein the controlling comprises inhibition of PAR-1 signaling pathway.

4. The method of claim 1 wherein the PAR signaling pathway is controlled by controlling phosphorylation of tissue factor cytoplasmic domain in said tissue.

5. The method of claim 1 wherein the PAR signaling pathway is controlled by administering to a mammal suffering from a pathological neovascularization disease state a therapeutically effective amount of a PAR signaling pathway inhibitor.

6. The method of claim 5 wherein the PAR signaling pathway inhibitor is a TF-VIIa signaling inhibitor.

7. The method of claim 6 wherein the TF-VIIa signaling inhibitor is selected from the group consisting of an active-site inhibited factor VIIa (VIIai), a nematode anticoagulant peptide c2 (NAPc2), an antibody specific for factor VIIa, an antibody specific for tissue factor-factor VIIa complex (TF-VIIa complex), and a combination thereof.

8. The method of claim 5 wherein the PAR signaling pathway inhibitor is a PDGF receptor β signaling inhibitor.

9. The method of claim 8 wherein the PDGF receptor β signaling inhibitor is an antibody specific for PDGF-BB.

10. The method of claim 3 wherein the PAR signaling pathway inhibitor is a tissue factor cytoplasmic domain phosphorylation inhibitor.

11. The method of claim 5 wherein the disease state is a cancer involving tumor development or an ischemic retinopathic disease.

12. The method of claim 1 wherein the mammal is a human.
